(19) United States
(12) Patent Application Publication
(10) Pub. No.: US 2018/0304023 A1
(43) Pub. Date: Oct. 25, 2018

(54) INJECTION SYRINGE

(71) Applicants: SALOME TKEBUCHAVA, NEWTON, MA (US); TENGIZ TKEBUCHAVA, NEWTON, MA (US)

(72) Inventors: SALOME TKEBUCHAVA, NEWTON, MA (US); TENGIZ TKEBUCHAVA, NEWTON, MA (US)

(21) Appl. No.: 15/920,894

(22) Filed: Mar. 14, 2018

Related U.S. Application Data
(60) Provisional application No. 62/472,632, filed on Mar. 17, 2017.

(51) Int. Cl. A61M 5/315; G01L 7/02

(52) U.S. Cl. CPC A61M 5/31511; A61M 2005/006; G01L 7/026

(57) ABSTRACT

An injection syringe device includes a tubular barrel, a plunger, and a piezo-chromic pressure indicator. The plunger has a distal tip and is designed to fit tightly and to reciprocate within the tubular barrel. Pulling the plunger out of the tubular barrel draws a liquid or a gas inside the tubular barrel in a space between the distal tip and a distal end of the tubular barrel and pushing the plunger into the tubular barrel expels the liquid or gas from the space. The piezo-chromic pressure indicator changes color to indicate pressure applied on the liquid or gas being expelled and/or target tissue being injected with the liquid or gas.

### CROSS REFERENCE TO RELATED CO-PENDING APPLICATIONS

This application claims the benefit of U.S. provisional application Ser. No. 62/472,632 filed Mar. 17, 2017 and entitled “IMPROVED INJECTION SYRINGE”, the contents of which are expressly incorporated herein by reference.

### FIELD OF THE INVENTION

The present invention relates to an improved injection syringe and in particular to an injection syringe that includes a pressure indicator and is used for injecting live cells and/or non-cell containing materials under controlled pressure into sensitive target tissues.

### BACKGROUND OF THE INVENTION

In several medical applications, injection syringes are used for injecting live cells, stem cells, blood, other biologics, or polymers into the bloodstream of a patient. Many of the injected cells are destroyed during the injection due to shear stresses and/or high injection pressure. Also, when the cells or non-cell containing materials are injected into an organ or other sensitive tissues, such as heart muscle, or liver, among others, a vigorous injection of any agents, particularly a viscous material like biopolymers, gels, and others, could damage the target tissue. In these applications it is critical to be able to control the pressure that is applied onto the plunger in order to avoid destroying the live cells or target tissues due to shear stresses and/or high pressure. It would also be desirable to have an indicator that indicates the level of the applied pressure on the live cells during the injection.

Furthermore, in many conventional syringes there is a dead space formed between the syringe hub and the tip of the needle and the material to be injected remains caught in this dead space and is not fully emptied. When the syringe is attached to a catheter or tube, the dead space increases. In some conventional syringes this dead space can be as high as 84 microliters. It would be desirable to have a syringe and an injection mechanism that reduces the dead space between the syringe hub and the needle and ensures that all injectate material is injected and flushed out of the syringe.

Furthermore, for drainage applications and blood drawing applications, it would be desirable to be able to sustain a negative pressure within the syringe without the risk of disengagement.

### SUMMARY OF THE INVENTION

An improved injection syringe includes a piezo-chromic pressure indicator that changes color to indicate increased pressure on the injectate and target tissues.

In general, in one aspect, the invention features an injection syringe device including a tubular barrel, a plunger, and a piezo-chromic pressure indicator. The plunger has a distal tip and is designed to fit tightly and to reciprocate within the tubular barrel. Pulling the plunger out of the tubular barrel draws a liquid or a gas inside the tubular barrel in a space between the distal tip and a distal end of the tubular barrel and pushing the plunger into the tubular barrel expels the liquid or gas from the space. The piezo-chromic pressure indicator changes color to indicate pressure applied on the liquid or gas being expelled and/or target tissue being injected with the liquid or gas.

Implementations of this aspect of the invention may include one or more of the following features. The device further includes a syringe hub attached to the distal end of the tubular barrel and a needle mounted onto the syringe hub. The device further includes a tube or a nozzle attached to the distal end of the tubular barrel. The liquid may be one of live cells, stem cells, blood, biologics, drugs, polymers, radiocontrast agent containing liquids, or combinations thereof. The distal tip of the plunger comprises the piezo-chromic pressure indicator and applying pressure onto a proximal end of the plunger causes the distal tip to change color thus indicating a measure of the applied pressure. The distal tip comprises an elastomeric compound and a piezo-chromic dye that is mixed into the elastomeric compound. The distal tip comprises an elastomeric compound that is coated with a piezo-chromic dye. The plunger comprises an elastomeric compound and a piezo-chromic dye that is mixed into the elastomeric compound. The elastomeric compound may be one of fluoropolymers, neoprene, nitrile, butyl, silicone, fluorocarbon, or tetrafluoroethylene-propylene. The piezo-chromic dye provides reversible or irreversible color changes under pressure and the color changes comprise one of from one distinct color to another distinct color, or from colorless to a distinct color, or from a distinct color to colorless. The piezo-chromic dye changes color from an original color to a new color at a defined applied pressure intensity and recovers the original color when the defined applied pressure intensity is removed. The piezo-chromic dye changes color from an original color to a new color at a first defined applied pressure intensity and recovers the original color at a second defined applied pressure intensity. The piezo-chromic dye changes color from an original color to a new color at a defined applied pressure intensity and the new color remains unchanged when the defined applied pressure intensity is removed. The piezo-chromic dye comprises one of triaryl imidazole dimer of bis-2,4,5-triaryl imidazole, bis-tetraaryl pyrrole, bianthrones, xanthylidene anthrone, dixanthylene, helianthrone, or mesonaphthobianthrone. The distal tip comprises a leading ring, a trailing ring and a main body between the leading ring and the trailing ring and the main body comprises a smaller diameter than the leading ring and the trailing ring. A first liquid is accumulated in a first space between the main body, the leading ring and the trailing ring and the tubular barrel, and a second liquid is accumulated in a second space between the leading ring and the distal end of the tubular barrel. The plunger further comprises fins extending sidewise from opposite sides of an outer surface of the plunger. The fins are inclined away from the distal tip of the plunger and wherein the tubular barrel further comprises a protrusion element extending horizontally from an inner surface or outer surface of a proximal end of the tubular barrel, and wherein the protrusion element is dimensioned to interfere with the fins and to prevent backward or forward motion of the plunger.

In general, in one aspect, the invention features a method for injecting a liquid or a gas into a target including the following: Providing an injection syringe comprising a tubular barrel and a plunger. The plunger comprises a distal tip and is designed to fit tightly and to reciprocate within the tubular barrel. Next, pulling the plunger out of the tubular barrel to draw a liquid or a gas inside the tubular barrel in a space between the distal tip and a distal end of the tubular barrel. Next, pushing the plunger into the tubular barrel to expel the liquid or gas from said space and into a target. A piezo-chromic pressure indicator changes color to indicate pressure applied on the liquid or gas being expelled and/or target being injected with said liquid or gas. The distal tip of the plunger comprises the piezo-chromic pressure indicator. Applying pressure onto a proximal end of the plunger causes the distal tip to change color thus indicating a measure of the applied pressure.

The details of one or more embodiments of the invention are set forth in the accompanying drawings and description below. Other features, objects, and advantages of the invention will be apparent from the following description of the preferred embodiments, the drawings and from the claims.

### BRIEF DESCRIPTION OF THE DRAWINGS

FIG. 1 a prior art injection syringe;

FIG. 2A-FIG. 2C depict an improved injection syringe at various stages of applying pressure to the proximal end of the plunger, according to this invention;

FIG. 3 depicts another aspect of the improved injection syringe according to this invention;

FIG. 4A to FIG. 4B depict forward and backward motion of the plunger, respectively, in another embodiment of an improved injection syringe according to this invention; and

FIG. 5 depicts another embodiment of improved injection syringe according to this invention.

### DETAILED DESCRIPTION OF THE INVENTION

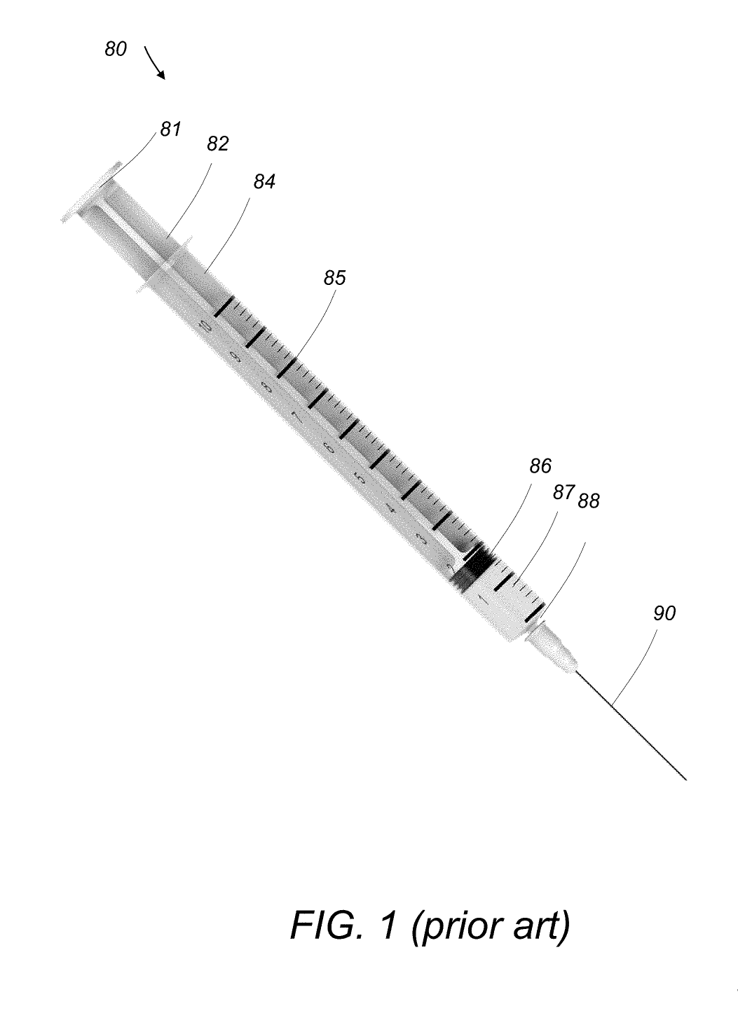

Referring to FIG. 1, a typical syringe 80 includes a plunger 82, a tubular barrel 84, a syringe hub 88, and a needle 90. The plunger 82 is designed to fit tightly and reciprocate within the barrel 82 and includes a distal sealing tip 86 and a proximal flat end 81. The tubular barrel 84 is made of transparent material such as glass or plastic and includes volume markings 85 on the cylindrical surface. Pulling the plunger 82 out of the barrel 84 allows the syringe to draw liquid or gas in the barrel space 87 between the sealing tip 86 and the syringe hub 88. Pushing the plunger 82 into the barrel 84 expels any liquid of gas from the space 87 between the sealing tip 86 and the syringe hub 88. Sealing tip 86 seals the barrel space 87 between the sealing tip 86 and the syringe hub 88 and the proximal flat end 81 is used for pushing the plunger 82 into the barrel 84 or for pulling the plunger 82 out of the barrel 84. Needle 90 is mounted onto the syringe hub 88 and is used to direct liquids or gases into and out of the barrel 84. Needle 90 has an elongated through opening that communicates with an opening in the syringe hub 88. In other embodiments syringe 80 is outfitted with a tube or a nozzle instead of a needle. Syringe 80 is used for administering drugs contained in the barrel space 87 between the sealing tip 86 and the syringe hub 88 or for drawing blood or other liquids. In some applications, syringe 80 is used for injecting live cells, stem cells, blood, or other biologics into the bloodstream of a patient. In these applications it is critical to be able to control the pressure that is applied onto the plunger 82 in order to avoid destroying of the live cells due to shear stresses and/or high pressure. It is also desirable to have a syringe and an injection mechanism that reduces the dead space between the syringe hub 88 and the needle 90 and ensures that all injectate material is injected and flushed out of the syringe and the needle. For drainage applications and blood drawing applications, it is also desirable to be able to sustain a negative pressure within the syringe without the risk of accidental disengagement.

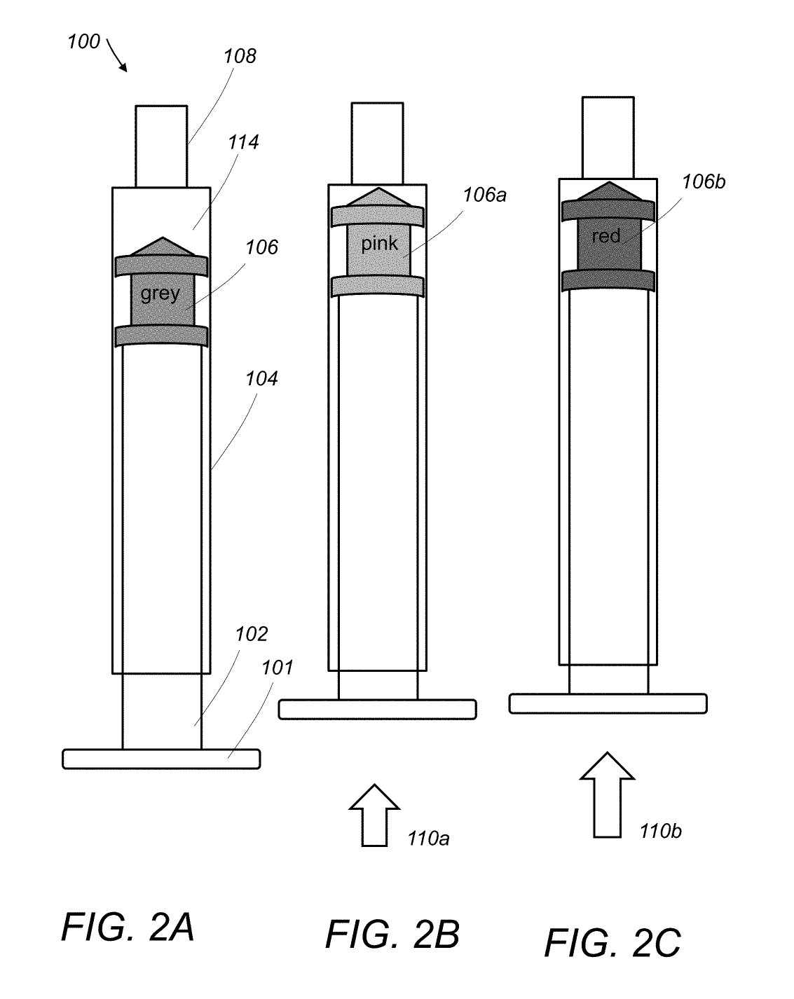

Referring to FIG. 2A-FIG. 2C, an improved syringe 100 according to this invention includes a plunger 102, a barrel 104, and a hub 108. The distal tip 106 of the plunger is made out of piezochromic materials that change color as a function of the applied pressure. As shown in FIG. 2A, tip 106 has initially a grey color when no pressure is applied onto the proximal end 101 of the plunger 102. When pressure 110a is applied to the proximal end 101, the distal tip 106 changes color to pink 106a, as shown in FIG. 2B. When a pressure 110b that is higher than pressure 110a is applied, the distal tip 106 changes color again to red 106b. The change in color of the distal tip 106 is used as an indicator of the pressure applied onto the biologics that are contained in space 114 of the barrel 104 and the goal is to maintain the pressure below a threshold that would destroy the live cells of the biologic compound. Tip 106 is made of an elastomeric compound and a piezochromic dye that is mixed into the elastomeric compound. In other embodiments, tip 106 is coated with the piezochromic material. In yet other embodiments, the piezochromic material is located in the main body of the plunger or the entire plunger is made of piezochromic material. Examples of elastomeric compounds include fluoropolymers, neoprene, nitrile, butyl, silicone, fluorocarbon, and tetrafluoroethylene-propylene, among others. The piezochromic dyes may provide reversible or irreversible color changes under pressure and the color changes may be from one distinct color to another distinct color or from colorless to a distinct color or from a distinct color to colorless. In one example the piezochromic material is provided by OliKrom of Pessac France. The OliKrom@PSoft version products change color at a defined pressure intensity and recover the original color when the applied pressure is removed. The OliKrom@PMemory version products change color at a defined pressure. P1, and recover the original color when the constraint drops below P2. The difference between P1 and P2 defines the memory effect, and allows specifying the history of the product (if a material has overpassed a threshold intensity of pressure). The OliKrom@POne version products change color at a defined pressure and remain unchanged when the constraint drops. In other embodiments, the piezochromic materials include piezochromic dyes disclosed in U.S. Pat. No. 5,501,945 These piezochromic dyes include one or more of triaryl imidazole dimer of bis-2,4,5-triaryl imidazole, bis-tetraaryl pyrrole, bianthrones, xanthylidene anthrone, dixanthylene, helianthrone, and mesonaphthobianthrone, among others.

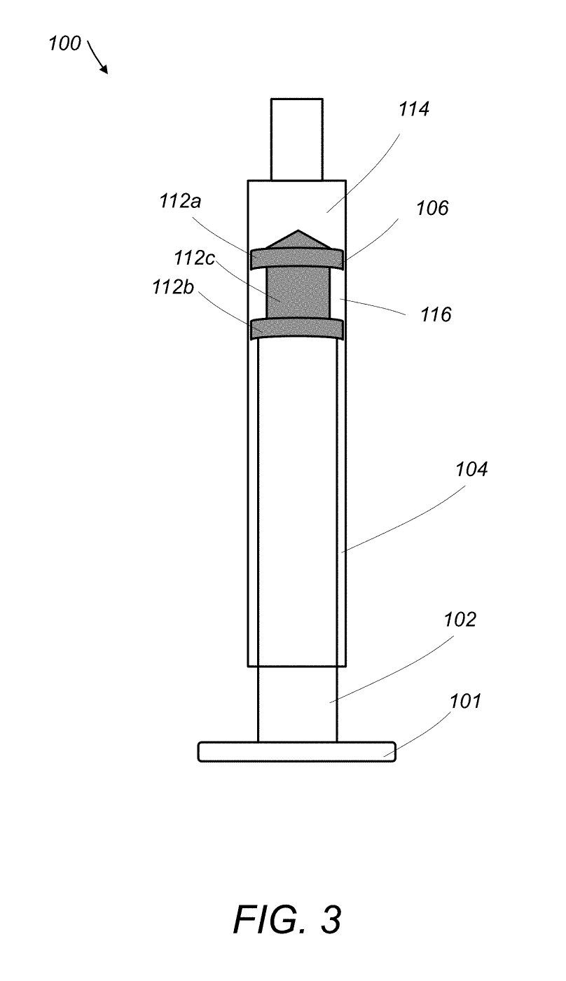

Referring to FIG. 3, the improved syringe 100 according to this invention further includes a distal tip 112 that has a leading ring 112a, a trailing ring 112b, and a main body 112c. The leading ring 112a seals the space 114 between the top of the barrel 104 and the tip of the plunger 102. The trailing ring 112b seals the space 116 between the body 112c and the barrel 104, as shown in FIG. 3. Initial aspiration of a first liquid such as water is accumulated in space 116 and once this space is filled a second liquid such as live cells is drawn into space 114 without mixing with the first liquid. When the liquids are dispensed, first the second liquid (i.e., live cells) from space 114 is injected and then the first liquid (i.e., water) from space 116 is injected. In case where the first liquid is water, the water is used to flush all the remaining live cells of the second liquid into the injected location. This distal tip design ensures that all injectate material is injected and flushed out of the syringe and the needle.

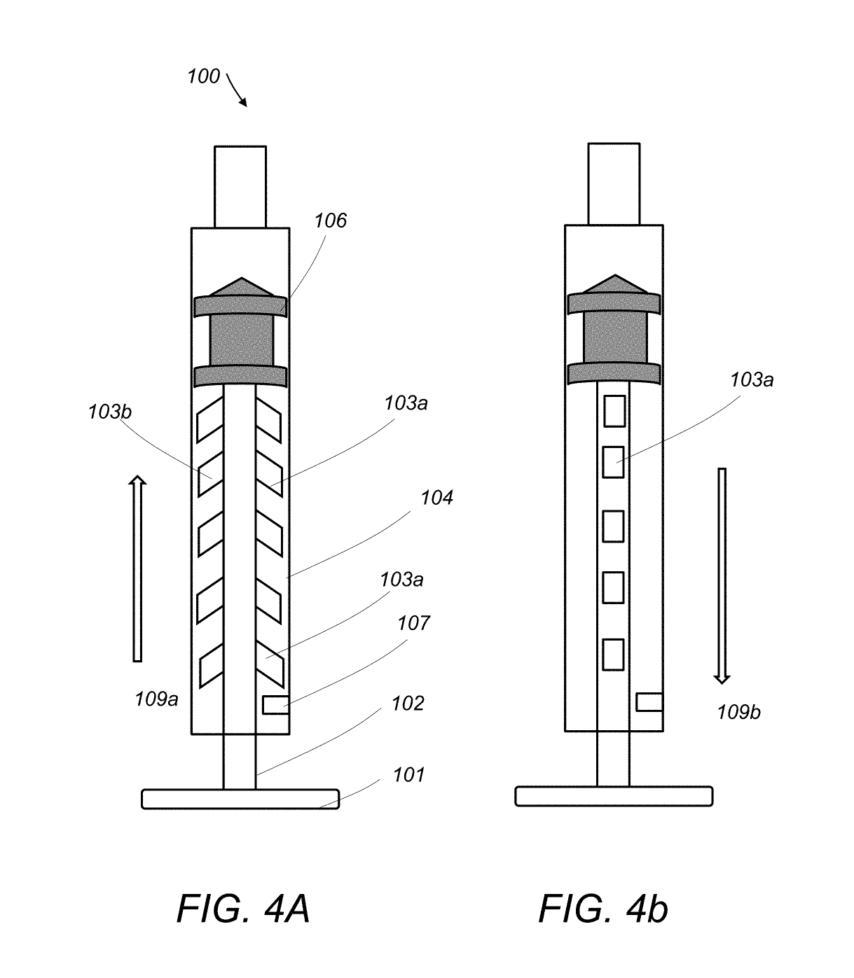

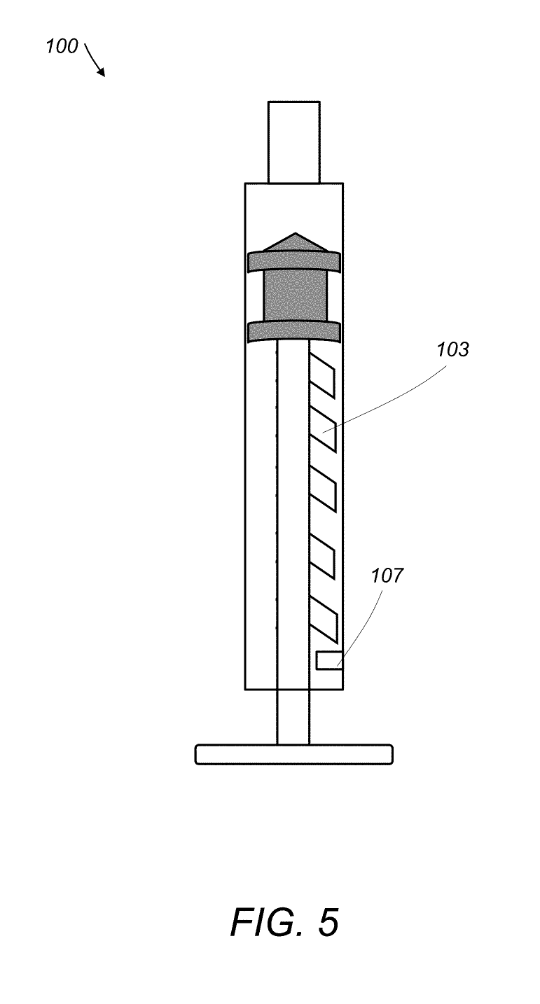

Referring to FIG. 4A-4B, the improved syringe 100 according to this invention further includes a plunger 102 with fins 103a, 103b extending sidewise from opposite sides of the plunger outer surface. Fins 103a, 103b are inclined away from the distal tip 106 and are used to slow down the forward or backward movement of the plunger 102 into or out the barrel 104 along direction 109a or direction 109b and to prevent backward movement of the plunger 102 along direction 109b. At the proximal end of the barrel 104 there is a protrusion element 107 that prevents backward or forward movement of the plunger 102 due to interference with the fins 103a, as shown in FIG. 4A. Forward motion of the plunger 102 along direction 109a is slowed down due to the stepwise interference between the fins 103a and the protrusion element 107. This arrangement protects the live cells in the injected liquid from experiencing an unintentional excessive pressure. Backward motion of the plunger 102 along direction 109b is stopped due to the stepwise interference between the fins 103a and the protrusion element 107. This arrangement enables sustaining a negative pressure within the syringe without the risk of accidental disengagement, which is useful for drainage applications and blood drawing applications. Rotating the plunger 102 by 90° degrees from the orientation of FIG. 4A disengages the fins 103a from the protrusion element 107 and allows the backward movement of the plunger 102 along direction 109b, as shown in FIG. 4B. The backward motion may be stepwise by rotating the plunger 102 back to 0° degrees from the orientation of FIG. 4A between each fin. Alternatively, the plunger 102 may be moved back at once, while it is held rotated 90° degrees from the orientation of FIG. 4A. In this embodiment, fins 103a and 103b extend from opposite sides of the outer surface of the plunger 102. In other embodiments, fins 103 extend only from one side of the outer surface of the plunger 102, as shown in FIG. 5. Protrusion element 107 is a cog or a tooth that is formed on an inner surface of the proximal end of the barrel 104.

Other embodiments include one or more of the following features. The plunger 102 is rotated by some degrees other than 90° degrees, such as 30°, 40°, 60° degrees, among others, in order to disengage or engage the fins. The entire plunger is made of piezochromic materials that change color as a function of the applied pressure. The plunger 102 is moved forward or backward via a spiral rotational motion. The spiral rotational movement prevents a speedy injection or aspiration in both the forward and the backward directions. The plunger 102 has a spiral carving and at the proximal portion of the barrel 104 there is a protrusion element on one or both inner sides or outer sides. This protrusion element interfaces with the spiral carving in the plunger 102 and this arrangement controls the forward or backward plunger movement.

Several embodiments of the present invention have been described. Nevertheless, it will be understood that various modifications may be made without departing from the spirit and scope of the invention. Accordingly, other embodiments are within the scope of the following claims.

What is claimed is:

1. An injection syringe device comprising:
a tubular barrel and a plunger;
wherein the plunger comprises a distal tip and is designed to fit tightly and to reciprocate within the tubular barrel and wherein pulling the plunger out of the tubular barrel draws a liquid or a gas inside the tubular barrel in a space between the distal tip and a distal end of the tubular barrel and pushing the plunger into the tubular barrel expels the liquid or gas from said space; and
a piezo-chromic pressure indicator that changes color to indicate pressure applied on said liquid or gas being expelled and/or target tissue being injected with said liquid or gas.

2. The device of claim 1, further comprising a syringe hub attached to the distal end of the tubular barrel and a needle mounted onto the syringe hub.

3. The device of claim 1, further comprising a tube or a nozzle attached to the distal end of the tubular barrel.

4. The device of claim 1, wherein the liquid comprises one of live cells, stem cells, blood, biologics, drugs, polymers, radiocontrast agent containing liquids, or combinations thereof.

5. The device of claim 1, wherein the distal tip of the plunger comprises said piezo-chromic pressure indicator and wherein applying pressure onto a proximal end of the plunger causes the distal tip to change color thus indicating a measure of the applied pressure.

6. The device of claim 5, wherein the distal tip comprises an elastomeric compound and a piezo-chromic dye that is mixed into the elastomeric compound.

7. The device of claim 5, wherein the distal tip comprises an elastomeric compound that is coated with a piezo-chromic dye.

8. The device of claim 1, wherein the plunger comprises an elastomeric compound and a piezo-chromic dye that is mixed into the elastomeric compound.

9. The device of claim 6, wherein the elastomeric compound comprises one of fluoropolymers, neoprene, nitrile, butyl, silicone, fluorocarbon, or tetrafluoroethylene-propylene.

10. The device of claim 6, wherein the piezo-chromic dye provides reversible or irreversible color changes under pressure and the color changes comprise one of from one distinct color to another distinct color, or from colorless to a distinct color, or from a distinct color to colorless.

11. The device of claim 6, wherein the piezo-chromic dye changes color from an original color to a new color at a defined applied pressure intensity and recovers the original color when the defined applied pressure intensity is removed.

12. The device of claim 6, wherein the piezo-chromic dye changes color from an original color to a new color at a first defined applied pressure intensity and recovers the original color at a second defined applied pressure intensity.

13. The device of claim 6, wherein the piezo-chromic dye changes color from an original color to a new color at a defined applied pressure intensity and the new color remains unchanged when the defined applied pressure intensity is removed.

14. The device of claim 6, wherein the piezo-chromic dye comprises one of triaryl imidazole dimer of bis-2,4,5-triaryl imidazole, bis-tetraaryl pyrrole, bianthrones, xanthylidene anthrone, dixanthylene, helianthrone, or mesonaphthobianthrone.

15. The device of claim 1, wherein the distal tip comprises a leading ring, a trailing ring and a main body between the leading ring and the trailing ring and wherein the main body comprises a smaller diameter than the leading ring and the trailing ring.

16. The device of claim 15, wherein a first liquid is accumulated in a first space between the main body, the leading ring and the trailing ring and the tubular barrel, and a second liquid is accumulated in a second space between the leading ring and the distal end of the tubular barrel.

17. The device of claim 1, wherein the plunger further comprises fins extending sidewise from opposite sides of an outer surface of the plunger.

18. The device of claim 17, wherein the fins are inclined away from the distal tip of the plunger and wherein the tubular barrel further comprises a protrusion element extending from a surface of a proximal end of the tubular barrel, and wherein the protrusion element is dimensioned to interfere with the fins and to prevent backward motion of the plunger.

19. A method for injecting a liquid or a gas into a target comprising:
providing an injection syringe comprising a tubular barrel and a plunger, wherein the plunger comprises a distal tip and is designed to fit tightly and to reciprocate within the tubular barrel;
pulling the plunger out of the tubular barrel to draw a liquid or a gas inside the tubular barrel in a space between the distal tip and a distal end of the tubular barrel;
pushing the plunger into the tubular barrel to expel the liquid or gas from said space and into a target; and
providing a piezo-chromic pressure indicator that changes color to indicate pressure applied on said liquid or gas being expelled and/or target being injected with said liquid or gas.

20. The method of claim 19, wherein the distal tip of the plunger comprises said piezo-chromic pressure indicator and wherein applying pressure onto a proximal end of the plunger causes the distal tip to change color thus indicating a measure of the applied pressure.
